(19) United States
(12) Patent Application Publication
(10) Pub. No.: US 2024/0058542 A1
(43) Pub. Date: Feb. 22, 2024

(54) OVAL PLUNGER PISTON WITHO-RING PRESSURE EQUALIZING CHANNELS FOR IMPROVED LEAK PERFORMANCE

(71) Applicant: Becton, Dickinson and Company, Franklin Lakes, NJ (US)

(72) Inventors: Nicholas P. ANDERSON, Whitman, MA (US); Nitish Kumar Varma KUNAPARAJU, Andover, MA (US)

(73) Assignee: Becton, Dickinson and Company, Franklin Lakes, NJ (US)

(21) Appl. No.: 18/266,907

(22) PCT Filed: Dec. 9, 2021
(86) PCT No.: PCT/US2021/062596

§ 371 Date: Jun. 13, 2023

Related U.S. Application Data
(60) Provisional application No. 63/125,603, filed on Dec. 15, 2020.

(51) Int. Cl. A61M 5/315

(52) U.S. Cl. CPC A61M 5/31511

(57) ABSTRACT

Disclosed is a plunger piston for use in a syringe and especially in a micro-dosing syringe pump application. The plunger piston comprises a front face opposite a rear face and at least one annular groove positioned between the front face and the rear face. The plunger piston includes a plurality of channels extending from the front face into the groove, the channels providing communication between the front face and the groove. An O-ring is received in the groove and the channels serve to equalize pressures around a first side of the O-ring to improve sealing performance and micro-dosing accuracy of a syringe or a micro-dosing syringe pump utilizing the plunger piston.

### CROSS-REFERENCE TO RELATED APPLICATIONS

This application claim priority under 35 USC § 119(e) from U.S. Provisional Patent Applications No. 63/125,603 filed Dec. 15, 2020, the content of which (including all attachments filed therewith) is hereby incorporated by reference in its entirety.

### FIELD OF THE DISCLOSURE

This present disclosure relates generally to piston plungers for drug delivery devices, and more particularly to a piston plunger having pressure equalizing channels to equalize pressure on an O-ring associated with the piston plunger.

### BACKGROUND OF THE DISCLOSURE

This section provides background information which is not necessarily prior art to the inventive concepts associated with the present disclosure.

Syringes are commonly used to dispense medicaments such as insulin, antibiotics, vaccines, chemotherapy drugs, pain relievers and many other medications. A common design includes a barrel or reservoir having a dispensing end for receipt of a needle or other connection and opposite it an open end for receipt of a plunger mechanism to push the fluid medicament out of the barrel and through the dispensing end. The plunger mechanism in the most basic form includes a plunger piston at one end and a location for a drive mechanism at an opposite end with a shaft in between. The drive mechanism, in the simplest form, can be a user's thumb to push the plunger mechanism into the barrel and dispense the medicament. In a micro-dosing syringe pump, the drive mechanism can involve an actual motor, which drives movement of the plunger piston into the barrel to dispense the medicament. Such syringe designs are well known to one of skill in the art. The plunger piston usually includes at least one annular groove or gland for receipt of an O-ring to provide a seal between the piston plunger and an inner wall of the barrel so that as the plunger piston is advanced into the barrel the fluid medicament in the barrel exits the dispensing end and does not get behind the plunger piston.

A common issue in designing a plunger piston is the need to balance the sealing pressure forces of the O-ring against the frictional forces between the O-ring and the inner wall of the barrel. These issues arise because in this environment the sealing needs to be a dynamic sealing as opposed to a static sealing. The O-ring must be designed to both seal the gap between the plunger piston and the inner wall of the barrel and still allow for smooth movement of the plunger piston so that accurate dosing can occur. The two forces are directly opposed to each other and can make it difficult to achieve the appropriate balance. In addition, the shape of the O-ring needs to be controlled in a defined manner under movement of the plunger piston to prevent uneven distortion that can exacerbate either of these issues. In conventional designs, one needed to accept a loss of power caused by an increase in frictional forces caused by uneven deformations of the O-ring. These occur when the pressures against the O-ring caused by advancement of the plunger mechanism are not evenly distributed around the entire O-ring. One conventional solution for other dynamic sealing applications was to provide an annular groove having a width such that there was a gap between the O-ring and the sides of the groove. In such a design, the O-ring can be biased toward one of the sidewalls of the groove to equalize pressures. This design, however, raises other issues in terms of insufficient sealing properties, the need to carefully balance the gap width and O-ring size and the looseness of the O-ring in the groove.

It is desirable to provide a solution that minimizes any gap between the sidewalls of the annular groove and the O-ring while still providing excellent sealing properties and accurate dosing from a syringe.

### SUMMARY OF THE DISCLOSURE

This section provides a general summary of the present disclosure and is not intended to be interpreted as a comprehensive disclosure of its full scope or all features, aspects and objectives.

One aspect of the present disclosure is to provide a plunger piston comprising: a front face opposite a rear face and at least one annular groove positioned between the front face and the rear face; and a plurality of channels extending from the front face into the groove, the channels providing communication between the front face and the groove.

In another aspect the present disclosure is a syringe comprising: a barrel and a plunger piston received in the barrel; the plunger piston comprising a front face opposite a rear face and at least one annular groove positioned between the front face and the rear face; and a plurality of channels extending from the front face into the groove, the channels providing communication between the front face and the groove.

These and other features and advantages of this disclosure will become more apparent to those skilled in the art from the detailed description herein. The drawings that accompany the detailed description are described below.

### BRIEF DESCRIPTION OF THE DRAWINGS

The drawings described herein are for illustrative purposes only of selected aspects and not all implementations, and are not intended to limit the present disclosure to only that actually shown. With this in mind, various features and advantages of example aspects of the present disclosure will become apparent to one possessing ordinary skill in the art from the following written description and appended claims when considered in combination with the appended drawings, in which:

FIG. 1 is a partial cross-sectional view of a conventional plunger piston having an O-ring and located inside a reservoir, the top panel shows a view with an initial low static pressure applied and the bottom panel shows the same view during application of greater dynamic pressure and the deformation of the O-ring;

FIG. 2 is a partial cross-sectional view of a plunger piston designed in accordance with the present disclosure;

FIG. 3 is a top view of a plunger piston designed in accordance with the present disclosure;

FIG. 4 is a bottom view of a plunger piston designed in accordance with the present disclosure;

FIG. 5 is a side view of a plunger piston designed in accordance with the present disclosure;

FIG. 6 is another side view of a plunger piston designed in accordance with the present disclosure;

FIG. 7 is a top view of a plunger piston designed in accordance with the present disclosure and an O-ring positioned on the plunger piston;

FIG. 8A is a schematic diagram showing a partial cross-sectional view of a plunger piston not in accordance with the present disclosure; and

FIG. 8B is a schematic diagram showing a partial cross-sectional view of a plunger piston in accordance with exemplary embodiments of the present disclosure and certain pressure and sealing benefits of the plunger piston according to exemplary implementations.

### DETAILED DESCRIPTION OF THE DISCLOSURE

In the following description, details are set forth to provide an understanding of the present disclosure.

For clarity purposes, example aspects are discussed herein to convey the scope of the disclosure to those skilled in the relevant art. Numerous specific details are set forth such as examples of specific components, devices, and methods, in order to provide a thorough understanding of various aspects of the present disclosure. It will be apparent to those skilled in the art that specific details need not be discussed herein, such as well-known processes, well-known device structures, and well-known technologies, as they are already well understood by those skilled in the art, and that example embodiments may be embodied in many different forms and that neither should be construed to limit the scope of the disclosure.

The terminology used herein is for the purpose of describing particular example aspects only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or feature is referred to as being “on,” “engaged to,” “connected to,” “coupled to” “operably connected to” or “in operable communication with” another element or feature, it may be directly on, engaged, connected or coupled to the other element or layer, or intervening elements or features may be present. In contrast, when an element is referred to as being “directly on,” “directly engaged to,” “directly connected to,” or “directly coupled to” another element or feature, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly and expressly indicated by the context. Thus, a first element, component, region, layer or section discussed below could be termed a second element, component, region, layer or section without departing from the teachings of the example embodiments.

For purposes of description herein, the terms “upper,” “lower,” “right,” “left,” “rear,” “front,” “vertical,” “horizontal,” and derivatives thereof shall relate to the disclosure as oriented in the FIGS. However, it is to be understood that the present disclosure may assume various alternative orientations and step sequences, except where expressly specified to the contrary. It is also to be understood that the specific devices and processes illustrated in the attached drawings, and described in the following specification are exemplary aspects of the inventive concepts defined in the appended claims. Hence, specific dimensions and other physical characteristics relating to the aspects disclosed herein are not to be considered as limiting, unless the claims expressly state otherwise.

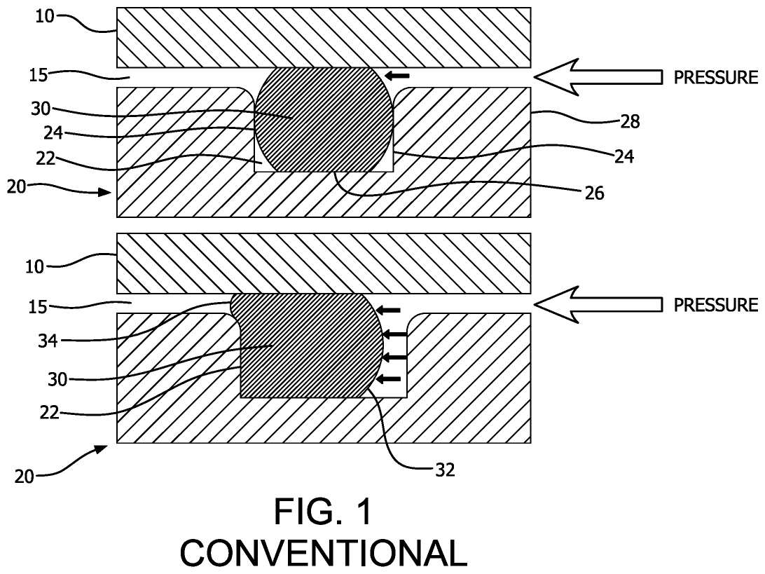

Referring in more detail to the drawings, FIG. 1 illustrates a partial cross-sectional view of a conventional system in two conditions, in a top panel and a bottom panel, as discussed herein. In the top panel, a partial cross-sectional view of a reservoir wall 10 is shown and a partial cross-sectional view of a plunger piston 20 having a front face 28 is shown. Throughout the present specification and claims the terms reservoir and reservoir wall can be substituted by a syringe barrel and a syringe barrel wall, respectively. The plunger piston 20 includes an annular groove 22, also known as an O-ring gland, for receipt of an O-Ring 30. The groove 22 includes a pair of opposed sidewalls 24 and a bottom 26. As can be seen there is an annular gap 15 located between the outside of the plunger piston 20 and the inside of the reservoir wall 10. The O-ring 30 is typically sized to contact a portion of the sidewalls 24 and the bottom 26 of the groove 22 along with an inner portion of the reservoir wall 10 when the plunger piston 20 is static at rest as shown in the top panel. Also shown is the direction of the pressure that would be exerted on the front face 28 of the plunger piston 20 and the O-ring 30 by a fluid or medicament (not shown) in the reservoir when the plunger piston 20 is moved longitudinally in one direction in the reservoir, here to the right side of the figure, against the fluid. In the top panel, the plunger piston 20 is at rest and minimal pressure is exerted on the plunger piston 20 and the O-ring 30.

In the bottom panel of FIG. 1 the plunger piston 20 is shown moving against the fluid (not shown) and the pressure exerted by the fluid is shown as arrows. As this movement occurs, the pressure is exerted through the gap 15 on a first side 32 of the O-ring 30. The pressure deforms the O-ring 30 causing it to move away from the sidewall 24 adjacent to a first side 32 of the O-ring facing the pressure, which leads a second side 34 of the O-ring 30 to deform against an adjacent sidewall 24 of the groove 22 and to deform into the gap 15 as shown. The O-ring 30 thus acts to seal the gap 15 between the reservoir wall 10 and the plunger piston 20 as the plunger piston 20 is moved longitudinally in the reservoir. As discussed herein, one issue that arises with this system is that the pressure is not always applied equally on the entire first side 32 of the O-ring 30 which can lead to localized non-uniform deformation of the O-ring 30, wear, failure in sealing and faulty dosing with micro-dosing syringe pump systems. The wear can come from excess friction caused by unbalanced application of the pressure on the first side 32 of the O-ring 30. This friction can also lead to issues with micro-dosing accuracy. It can be difficult to balance the size of the O-ring 30 relative to the size of the groove 22 and the groove 22 depth, width and shape and other groove 22 characteristics to provide for smooth movement of the plunger piston 20 and good sealing of the gap 15. The present disclosure is directed to overcoming these issues and finds special use in applications such as micro-dosing syringe pump systems such as for dosing of insulin, antibiotics, vaccines, chemotherapy drugs, and pain relievers, for example. These sorts of micro-dosing syringe pump systems need to have smooth movement and high sealing ability even when the plunger piston 20 is moved in very small increments.

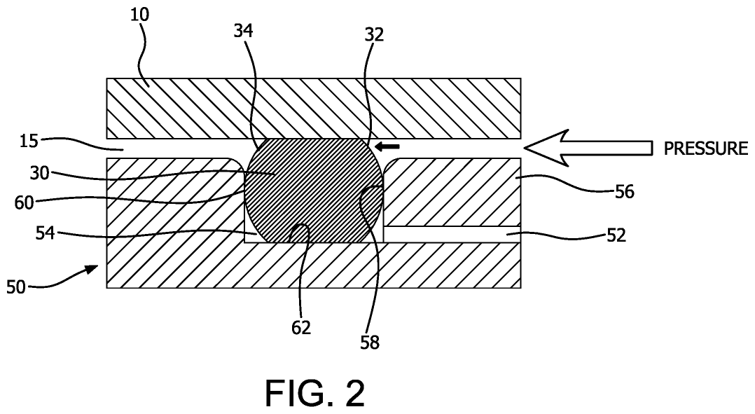

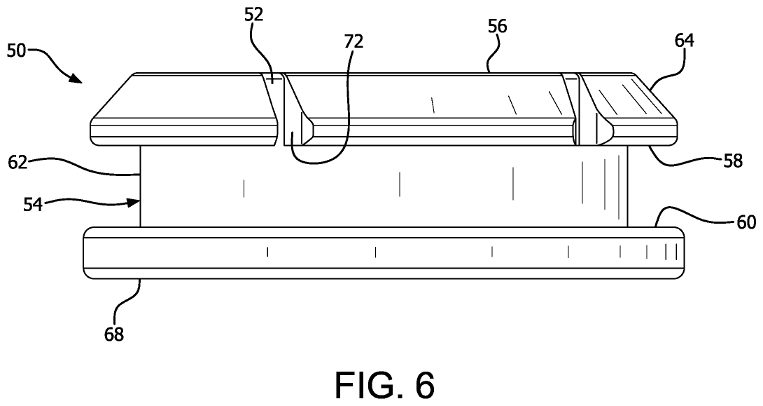

As shown in FIG. 2 a plunger piston 50 according to the present disclosure comprises a groove 54 for accommodating an O-ring 30 and includes a plurality of channels 52 in a front face 56 of the plunger piston 50. The channels 52 extend from the front face 56 to the groove 54 and pass through a front sidewall 58 of the groove 54, which accommodates the O-ring 30. The groove 54 has a front sidewall 58, a rear sidewall 60 and a bottom 62. The plurality of channels 52 allow the pressure of a fluid to be exerted not only through the gap 15 but also through the channels 52 at a plurality of points in the front face 56 of the plunger piston 50. These channels 52 equalize the pressure of the fluid around the entire first side 32 of the O-ring 30. This means the O-ring 30 can be better sized to reduce friction between the O-ring 30 and the inner side of the reservoir wall 10 while still providing excellent sealing characteristics. The present design also eliminates the need to have a groove 54 width between the sidewalls, 58 and 60, which provides a gap between the O-ring 30 and the sidewalls, 58 and 60. If desired for other reasons such a gap can be incorporated, however, it is not necessary. The pressure equalization eliminates the possibility of non-uniform deformation of the O-ring 30 and spreads the pressure uniformly around the first side 32 of the O-ring 30. The reduced friction increases the accuracy of the dosing especially when the plunger piston 50 is used in a micro-dosing syringe pump system. The number of channels 52, their size and placement around the front face 56 of the plunger piston 50 can be varied as desired to provide for uniform application of pressure to the first side 32 of the O-ring 30. Preferably, the channels 52 are equally spaced from each other around an outer rim 64 of the front face 56 to fully equalize the pressure against the first side 32 of the O-ring 30. Preferably, the depth of the channel 52 extends from the outer diameter of the outer rim 64 of the front face 56 to the bottom of the groove 54 as shown in the Figures, see especially FIG. 6. The plunger piston 50 can be formed from a variety of materials including plastics, elastomers and polymeric materials as is known in the art.

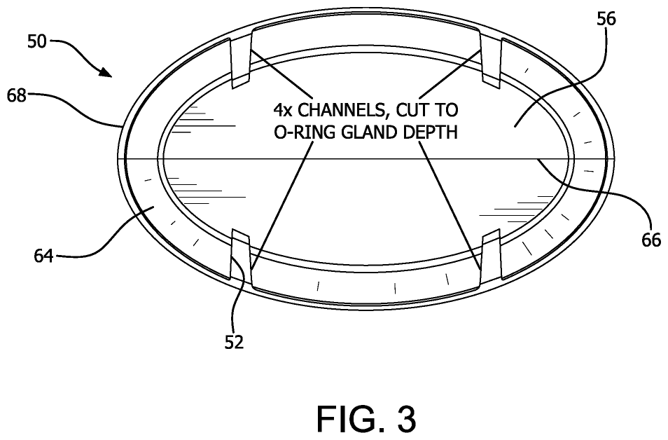

FIG. 3 is a top view of a plunger piston 50 designed in accordance with the present disclosure. The front face 56 is shown with four channels 52 cut into the front face 56. The channels 52 are shown as being generally perpendicular to a centerline 66 of the front face 56, however this is not necessary. The channels 52 can be at any orientation relative to a centerline 66 of the plunger piston 50. The number of channels 52 can also be varied as required to equalize the pressure on the first side 32 of the O-ring 30. As shown, preferably the channels 52 are cut to a depth equal to the depth of the groove 54. Also in this embodiment the plunger piston 50 is shown as having an outer oval shape, however it could also be circular as known in the art. As would be known to one of skill in the art, when using a plunger piston 50 having an outer oval shape the inner portion of the reservoir wall 10 will also have a matching oval shape. The outer shape of the plunger piston 50 just needs to match that of the inner portion of the reservoir wall 10. An outer oval shape of the plunger piston 50 can provide benefits such as resistance to rotation of the plunger piston 50 as it moves in the reservoir along the reservoir wall 10 and reduced reservoir wall 10 deformation. The front face 56 has an outer rim 64 that is preferably wedged shaped, see especially FIG. 6. This wedge shape helps the plunger piston 50 to move smoothly against the fluid pressure in the reservoir. The outer rim 64 does not have to have a wedge shape for this inventive concept. In addition, as can be seen the diameter of the front face 56 oval is slightly less than that of a diameter of a rear face 68 oval of the plunger piston 50.

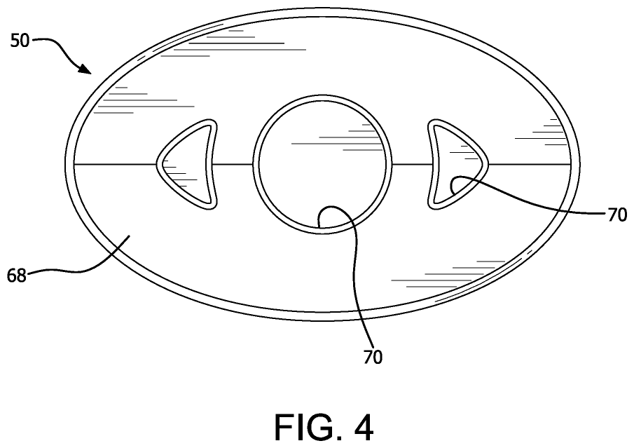

FIG. 4 is a bottom view of a plunger piston 50 designed in accordance with the present disclosure. As can be seen the rear face 68 of the plunger piston 50 is also oval shaped and includes at least one detent 70 and preferably a plurality of detents 70. The detent(s) 70 is/are used to engage a driver mechanism, not shown, to drive the plunger piston 50 in the reservoir as known to those of skill in the art. The driver mechanism generally includes a shaft and can comprise any of a number of designs including a straight shaft, a screw threaded shaft, a telescoping set of driver shafts, a scissors type driver and any other type of known driver mechanism for a plunger piston. The most common types in micro-dosing syringe pumps include threaded shafts and telescoping drivers, usually comprising a set of screw driven shafts.

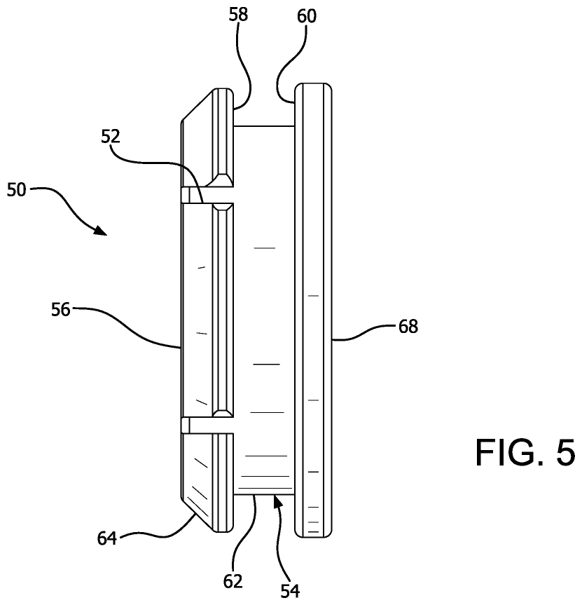

FIG. 5 and FIG. 6 are side views of a plunger piston 50 designed in accordance with the present disclosure. As can be seen the outer rim 64 of the front face 56 has a wedge 72 as can been seen in one of the channels 52. This shape is advantageous in distributing the pressure over the entire front face 56 and into the channels 52; however, other shapes could be utilized. Preferably, as shown in FIG. 5 and FIG. 6, the channel 52 has a depth that extends to the bottom 62 of the groove 54. A thickness of the outer rim 64 between the front face 56 and the front sidewall 58 can be adjusted as required by the pressures expected to be experienced in the particular application. Likewise, a thickness between the rear sidewall 60 and the rear face 68 of the plunger piston 50 can be adjusted as needed to provide the appropriate stiffness and support for the O-ring 30 and the plunger piston 50.

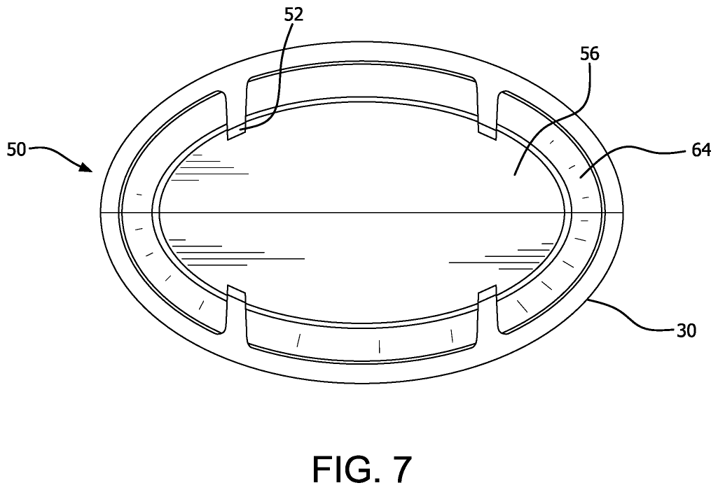

FIG. 7 is a top view of a plunger piston 50 designed in accordance with the present disclosure and the O-ring 30 positioned in the groove 54. The O-ring 30 has an outer diameter that is greater than that of the outer rim 64 and is sufficient for it to fill the gap 15 and contact an inner portion of the reservoir wall 10. The O-ring 30 can be formed from a rubber, elastomer and other typical O-ring materials. It is a typical O-ring designed with the appropriate characteristics of percent compression, percent stretch, percent volume fill for the groove 54 and gap 15, and other known characteristics dictated by the type of plunger piston 50, its size and the forces acted upon it in the reservoir during use. One of skill in the art can adjust these characteristics to meet the needs of the environment within which it will be used.

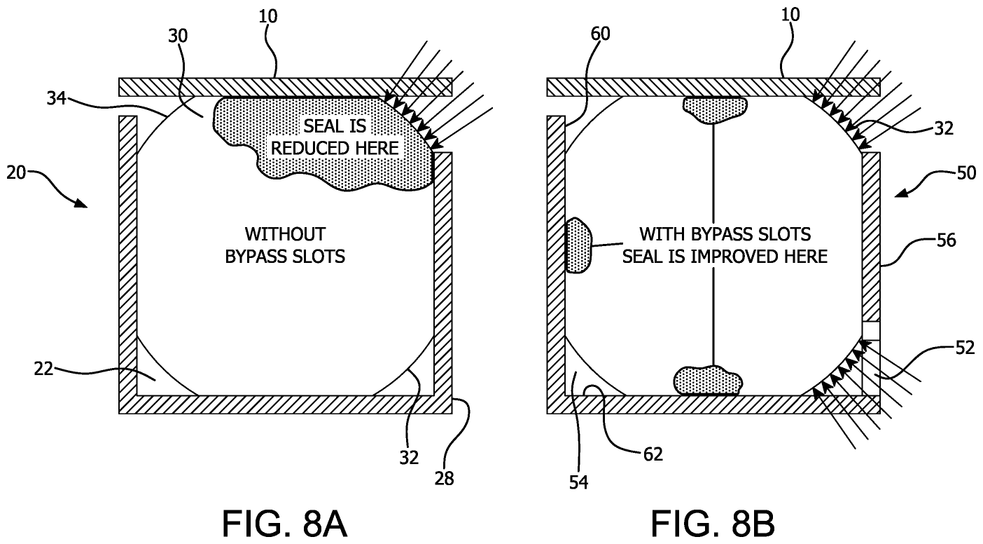

FIG. 8A is a schematic showing a partial cross-sectional view of a plunger piston 20 not in accordance with the present disclosure, on the left, next to FIG. 8B showing a partial cross-sectional view of a plunger piston 50 in accordance with exemplary embodiments of the present disclosure, on the right, and examples of the pressure and sealing benefits of a plunger piston 50 according to exemplary implementations of the disclosure. The pressure exerted by the fluid as the plunger piston 20 or 50 is moved in the reservoir is shown by the small arrows. As can be seen for the conventional plunger piston 20 the pressure is only coming through the gap 15 between the plunger piston 20 and the inner portion of the reservoir wall 10 and the seal is reduced in that area. The force does not expand over the entire first side 32 of the O-ring 30. By way of contrast,]a plunger piston 50 according to exemplary embodiments allows the force to be exerted through the gap 15 and the channels 52 allowing the force to evenly distribute over the entire first side 32 of the O-ring 30. Thus, better sealing can be provided, as shown by the circles, against the inner portion of the reservoir wall 10, the rear sidewall 60 and the bottom 62 of the groove 54.

As discussed herein the conventional designs required one to balance the dosing accuracy against the sealing effectiveness of the O-ring. This trade-off is eliminated in the inventive concept as it equalizes the pressure over the entire first side 32 of the O-ring 30 and this enhances dosing accuracy and sealing effectiveness in a single design.

The foregoing disclosure has been described in accordance with the relevant legal standards, thus the description is exemplary rather than limiting in nature. Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail. Accordingly, the scope of legal protection afforded this disclosure can only be determined by studying the following claims.

1. A plunger piston comprising:
a front face opposite a rear face and at least one annular groove positioned between said front face and said rear face, said annular groove including a pair of opposed sidewalls and a bottom;
an O-ring located in said groove, said O-ring being sized to contact at least a portion of said opposed sidewalls and said bottom; and
a plurality of channels extending from said front face into said groove, said channels providing communication between said front face and said O-ring to equalize a pressure on the O-ring when the front face of the plunger piston is moved against a fluid.

2. The plunger piston as recited in claim 1 wherein said front face includes an outer rim and wherein said channels are located in said outer rim.

3. The plunger piston as recited in claim 2, wherein said channels have a depth that extends from said outer rim to a bottom of said groove.

4. The plunger piston as recited in claim 2, wherein said outer rim has a wedge shape.

5. The plunger piston as recited in claim 1, wherein said plunger piston has an oval shape.

6. The plunger piston as recited in claim 5 wherein said channels are oriented perpendicular to a centerline of said oval shape.

7. The plunger piston as recited in claim 1, wherein said front face has a diameter that is smaller than a diameter of said rear face.

8. The plunger piston as recited in claim 1, wherein said rear face includes at least one detent.

9. (canceled)

10. The plunger piston as recited in claim 1 wherein said O-ring has an outer diameter that is greater than an outer diameter of said front face.

11. A syringe comprising:
a barrel and a plunger piston received in said barrel:
said plunger piston comprising a front face opposite a rear face and at least one annular groove positioned between said front face and said rear face, said annular groove including a pair of opposed sidewalls and a bottom;
an O-ring located in said groove, said O-ring being sized to contact at least a portion of said opposed sidewalls and said bottom; and
a plurality of channels extending from said front face into said groove, said channels providing communication between said front face and said O-ring to equalize a pressure on the O-ring when the front face of the plunger piston is moved against a fluid.

12. The syringe as recited in claim 11 wherein said front face includes an outer rim and wherein said channels are located in said outer rim.

13. The syringe as recited in claim 12, wherein said channels have a depth that extends from said outer rim to a bottom of said groove.

14. The syringe as recited in claim 12, wherein said outer rim has a wedge shape.

15. The syringe as recited in claim 11, wherein said plunger piston has an oval shape and wherein said barrel has an inner wall with a matching oval shape.

16. The syringe as recited in claim 15 wherein said channels are oriented perpendicular to a centerline of said oval shape.

17. The syringe as recited in claim 11, wherein said front face has a diameter that is smaller than a diameter of said rear face.

18. The syringe as recited in claim 11, wherein said rear face includes at least one detent.

19. (canceled)

20. The syringe as recited in claim 11 wherein said O-ring has an outer diameter that is greater than an outer diameter of said front face.
